(19) United States
(12) Patent Application Publication
(10) Pub. No.: US 2009/0023834 A1
(43) Pub. Date: Jan. 22, 2009

(54) METHOD FOR TREATING POLYPROPYLENE TEXTILES

(76) Inventors: Ralf Norenberg, Ludwigshafen (DE); Manfred Blauth, Ludwigshafen (DE); Jurgen Reichert, Limburgerhof (DE)

Correspondence Address: OBLON, SPIVAK, MCCLELLAND MAIER & NEUSTADT, P.C., 1940 DUKE STREET, ALEXANDRIA, VA, 22314, US

(21) Appl. No.: 12/162,130

(22) PCT Filed: Jan. 30, 2007
(86) PCT No.: PCT/EP07/50860

§ 371 Date: Jul. 25, 2008

(30) Foreign Application Priority Data
Feb. 9, 2006 (EP) 06101447.8

(51) Int. Cl. A61F 13/15; C08L 31/06; C08L 75/00

(52) U.S. Cl. 523/105; 524/556; 524/589

(57) ABSTRACT

A process for treating a polypropylene textile comprises treating the polypropylene textile with an emulsifier-free aqueous formulation comprising
(a) at least one silicone compound having at least one hydrophilic group or
(b) at least one ethylene copolymer obtainable by copolymerization of ethylene with at least one ethylenically unsaturated mono- or dicarboxylic acid or anhydride.

The present invention relates to a process for treating a polypropylene textile, which comprises treating the polypropylene textile with an emulsifier-free aqueous formulation comprising
(a) at least one silicone compound having at least one hydrophilic group or
(b) at least one ethylene copolymer obtainable by copolymerization of ethylene with at least one ethylenically unsaturated mono- or dicarboxylic acid or anhydride.

The present invention further relates to polypropylene textiles treated by the process of the present invention and to their use. The present invention further relates to aqueous formulations comprising
(a) at least one silicone compound having at least one hydrophilic group or
(b) at least one ethylene copolymer obtainable by copolymerization of ethylene with at least one ethylenically unsaturated mono- or dicarboxylic acid or anhydride.

The present invention further relates to the use of silicone compounds having at least one NH group or at least one alkylene oxide unit for hydrophilicizing polypropylene textiles.

Polypropylene is an inherently bacteria-hostile and inexpensive material for manufacturing textiles such as for example fibers and fabrics, in particular fiber material, and therefore is used in many functional textiles, for example in diaper nonwovens and as an addition to concrete for the purpose of fire protection. Using polypropylene in concrete fire protection makes use of the fact that polypropylene depolymerizes at temperatures above 200° C. to form gaseous propylene, which rapidly escapes, creating passageways along which water vapor, which in the event of a fire can lead to explosive spalling of the concrete structure at about 300° C., can dissipate.

The hydrophobicity of polypropylene is undesirable in many of the aforementioned applications. Therefore, polypropylene for diaper manufacture, for example, is hydrophilicized (rendered hydrophilic) by means of a sparingly water-soluble surface-active substance. The small amounts of these surface-active substances dissolved off in use harbor the a risk of skin irritation.

The aforementioned impermanence of the hydrophilic finish is also the reason why today the uniform distribution of polypropylene in concrete is still a substantial challenge. True, a plasma treatment would be a possibility for polypropylene fibers, but it is difficult to stabilize any satisfactory hydrophilicity on the part of the polypropylene fiber surface for any length of time, for example days.

The present invention has for its object to provide a process whereby polypropylene is simple to render very durably hydrophilic and which if possible avoids the abovementioned disadvantages such as potential skin irritation for example. The present invention further has for its object to provide formulations with which polypropylene is simple to render very durably hydrophilic. The present invention further has for its object to provide hydrophilicized polypropylene.

We have found that this object is achieved by the process defined at the beginning.

The process of the present invention proceeds from textiles composed of polypropylene, including textiles consisting of polypropylene and at least one further material, for example mixtures of polypropylene and cotton or polyester. Polypropylene herein shall comprehend not just homopolymers of propylene, but also such copolymers of propylene as comprise one or more olefins and preferably α-olefins such as for example 1-butene or 1-hexene or ethylene in interpolymerized form. Preferably, copolymers of propylene are propylene copolymers wherein at least 50% by weight of the interpolymerized comonomers is propylene, more preferably at least 70% by weight.

Polypropylene for the purposes of the present invention is preferably isotactic.

Textiles for the purposes of the present invention preferably comprise thread-shaped, i.e., linear structures, for example threads, fibers, filaments, microfilaments, microfibers, monofilaments, multifilaments, staple fibers, each textured or nontextured.

However, textiles for the purposes of the present invention may also comprise fabrics or sheetlike structures composed of polypropylene which are preferably obtainable by combination of one or more thread-shaped structures of polypropylene, examples being nonwovens, wovens, knits, laids, and blends, and which are producible using other similarly thread-shaped structures, for example synthetic fibers such as for example polyamide, polyethylene, polyester or polyacrylic, or natural fibers such as cotton for example. Preferably, however, textiles for the purposes of the present invention comprise fabrics or sheetlike structures or fibers or filaments each produced either from polypropylene or from mixtures of polyethylene and polypropylene.

In one embodiment of the present invention, the polypropylene textile to be treated has not been separately pretreated, i.e., neither hydrophilicized nor hydrophobicized, before the treatment of the present invention.

In one embodiment of the present invention, the textile to be treated has not been pretreated either by plasma treatment or by, for example, flash discharges.

According to the present invention, polypropylene textile is treated with an emulsifier-free aqueous formulation. Treating is here to be understood as meaning for example drenching, spraying, kiss-roll application and particularly padding. According to the present invention, treating is done more than once and preferably just once.

According to the present invention, polypropylene textile is treated with emulsifier-free aqueous formulation. This is herein to be understood as meaning that aqueous formulation with which polypropylene textile is treated has no low molecular weight emulsifier, i.e., no emulsifiers having molecular weights up to 400 g/mol, added to it, one embodiment comprising not adding emulsifiers having molecular weights of up to 500 g/mol. In the case of low molecular weight emulsifiers having molecular weight distributions, Mₙ is meant in each case.

Emulsifiers for the purposes of the present invention are surface-active anionic, cationic or nonionic compounds, examples being quaternary ammonium salts of C₈-C₄₀ fatty amines, C₈-C₄₀ fatty alcohol sulfates, C₈-C₄₀ fatty alcohol phosphates, C₈-C₄₀ fatty alcohol sulfonates, C₈-C₄₀ fatty alcohol phosphonates, sulfonates of C₈-C₃₀-alkyl-aromatics, from 15- to 150-tuply alkoxylated, for example ethoxylated or propoxylated, C₈-C₄₀ fatty alcohol.

There is one embodiment of the present invention where “emulsifier-free” is to be understood as meaning that altogether less than 0.1% by weight of above-characterized emulsifier is in the aqueous formulation used in the process of the present invention, preferably 0.001% to 0.01% by weight, based on the particular aqueous formulation.

Aqueous formulation used in the process of the present invention comprises
(a) at least one silicone compound having at least one hydrophilic group, herein also referred to as silicone compound (a), or
(b) at least one ethylene copolymer obtainable by copolymerization of ethylene with at least one ethylenically unsaturated mono- or dicarboxylic acid or anhydride, herein also referred to as ethylene copolymer (b).

Silicone compound (a) may comprise a quaternary ammonium group for example.

Silicone compounds (a) are preferably such compounds as are constructed of a plurality of Si(R¹)(R²)—O— units, so that Si—O— chains are formed, and as are terminally saturated either with OH groups or with further R¹ radicals. The R¹ and R² radicals may be different or mostly the same and mainly selected from C₆-C₁₄-aryl, particularly phenyl, or C₁-C₄-alkyl, preferably unbranched, and particularly methyl. Silicone compound (a) bears at least one hydrophilic group which may be present for example on one of the terminal or—if present—internal Si atoms of the Si—O chains described above. In one embodiment of the present invention, silicone compound (a) may comprise two or three hydrophilic groups per molecule, which may be different or preferably the same.

In one embodiment of the present invention, the hydrophilic group or groups are attached via a spacer to one of the terminal or—if present—internal Si atoms of the Si—O chains described above, for example via a C₂-C₂₀-alkylene spacer, which may be branched or preferably unbranched and in which one or more nonadjacent CH₂ groups may be replaced by oxygen atoms.

In one embodiment of the present invention, silicone compound (a) has a kinematic viscosity in the range from 100 to 100 000 m²/s, determined at 23° C.

In one embodiment of the present invention, silicone compound (a) comprises a silicone compound having at least one NH group or at least one C—OH group or at least one alkylene oxide unit per molecule.

For example, silicone compound (a) may comprise at least one NH group. Examples of NH groups are NH(C₁-C₁₀-alkyl) groups, in particular NH-methyl, NH-ethyl, NH(C₆-C₁₄-aryl) groups, particularly NH(C₆H₅), NH(C₇-C₂₀-aralkyl) groups, in particular NH-benzyl, NH(C₇-C₂₀-alkylaryl) groups, in particular NH-(para-tolyl), NH₂ groups, NH—CH₂—CH₂—NH₂, —CONH₂, NH—(CH₂)₃—NH₂.

In one embodiment of the present invention, silicone compound (a) may comprise at least one C—OH group, which may be for example an alcoholic OH group, or a COOH group. Preferably, at least one C—OH group in silicone compound (a) comprises a secondary or more preferably a primary C—OH group, for example a CH₂—OH group, a CH₂—CH₂—OH group or a CH₂—CH₂—CH₂—OH group.

In one embodiment of the present invention, silicone compound (a) may comprise at least one alkylene oxide unit per molecule, preferably at least 3 to 20 alkylene oxide units per molecule. Examples of alkylene oxide units are C₂-C₆-alkylene oxide units, for example propylene oxide units or butylene oxide units or preferably ethylene oxide units.

In one specific embodiment of the present invention, silicone compound (a) may be at least one compound obtained by reaction of excess of such silicone as comprises at least one Si—H group per molecule, preferably at least two Si—H groups per molecule, in the presence of a Pt-based catalyst with at least one compound of the general formula I

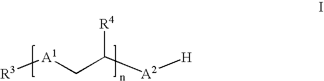

where
R³ is selected from C₁-C₆-alkyl, for example methyl, ethyl, n-propyl, iso-propyl, n-butyl, iso-butyl, sec-butyl, tert-butyl, n-pentyl, iso-pentyl, sec-pentyl, neo-pentyl, 1,2-dimethylpropyl, iso-amyl, n-hexyl, iso-hexyl, sec-hexyl, preferably C₁-C₄-alkyl such as methyl, ethyl, n-propyl, iso-propyl, n-butyl, iso-butyl, sec-butyl and tert-butyl;
   C₂-C₆-alkenyl, for example vinyl, 1-allyl, 2-allyl, 3-allyl, homoallyl, (O-hexenyl, ω-pentenyl and preferably vinyl or 3-allyl,
R⁴ in each occurrence is the same or different and independently selected from ethyl, methyl and particularly hydrogen,
A¹ and A² are different or preferably the same and selected from NH and O,
n is an integer in the range from 1 to 100 and preferably in the range from 2 to 50.

The excess of the compound of general formula I is based on equivalents of Si—H groups in the aforementioned silicone.

Suitable Pt-based catalysts are for example PtCl₄, H₂PtCl₆-6H₂O, platinum-olefin complexes, platinum supportated on finely divided silica gel or activated carbon or on alumina, or complexes of Pt with ethers, aldehydes, ketones, alkoxides, for example norbornadiene-platinum dichloride or 1,5-cyclooctadiene-platinum dichloride.

One specific embodiment of the present invention utilizes such silicone compounds (a) as is obtainable by reaction of the aforementioned silicone compound with one or more diisocyanates, preferably with one or more aliphatic or cycloaliphatic diisocyanates, for example 4, 4′-methylenedicyclohexyl diisocyanate, dodecamethylene diisocyanate, tetramethylene diisocyanate and particularly hexamethylene diisocyanate (HDI) and isophorone diisocyanate. Other suitable diisocyanates are aromatic diisocyanates such as 2,4-TDI, 2,6-TDI (tolylene diisocyanate).

One other specific embodiment of the present invention utilizes such silicone compounds (a) as is obtainable by reaction of the aforementioned silicone compound with one or more aliphatic, aromatic or cycloaliphatic diisocyanates and one or more diols, triols, diamines, triamines or polyamines, in particular aliphatic diols, triols, diamines, triamines or polyamines such as for example bis(dimethylaminopropyl)-amine. Preferred diols are aminodiols such as for example N-methyldiethanolamine, diethanolamine, N-(n-butyl)diethanolamine.

Silicone compounds of the aforementioned type and their preparation are described for example in WO 05/121218.

Aqueous formulation used in the process of the present invention may comprise at least one ethylene copolymer (b).

In one embodiment of the present invention, ethylene copolymer (b) comprises in interpolymerized form:

60% to 95% by weight and preferably 65% to 90% by weight of ethylene and 5% to 40% by weight and preferably 10% to 35% by weight of at least one ethylenically unsaturated mono- or dicarboxylic acid or anhydride, the weight % ages being based on ethylene copolymer (b).

Preferably, at least one ethylenically unsaturated carboxylic acid comprises a carboxylic acid of the general formula II

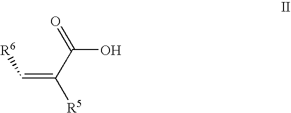

where
R⁵ is selected from hydrogen,
   C₁-C₁₀-alkyl, such as methyl, ethyl, n-propyl, iso-propyl, n-butyl, iso-butyl, sec-butyl, tert-butyl, n-pentyl, iso-pentyl, sec-pentyl, neo-pentyl, 1,2-dimethylpropyl, iso-amyl, n-hexyl, iso-hexyl, sec-hexyl, n-heptyl, n-octyl, n-nonyl, n-decyl; more preferably C₁-C₄-alkyl such as methyl, ethyl, n-propyl, iso-propyl, n-butyl, iso-butyl, sec-butyl and tert-butyl;
R⁶ is selected from hydrogen,
   C₁-C₁₀-alkyl, such as methyl, ethyl, n-propyl, iso-propyl, n-butyl, iso-butyl, sec-butyl, tert-butyl, n-pentyl, iso-pentyl, sec-pentyl, neo-pentyl, 1,2-dimethylpropyl, iso-amyl, n-hexyl, iso-hexyl, sec-hexyl, n-heptyl, n-octyl, n-nonyl, n-decyl; more preferably C₁-C₄-alkyl such as methyl, ethyl, n-propyl, iso-propyl, n-butyl, iso-butyl, sec-butyl and tert-butyl; COOH, COOCH₃, COOC₂H₅.

Most preferably, R⁵ is methyl or hydrogen and R⁶ hydrogen.

Suitable ethylenically unsaturated carboxylic anhydrides are in particular maleic anhydride and itaconic anhydride.

Ethylene copolymer (b) may comprise one or more further comonomers in interpolymerized form, namely up to 40% by weight and preferably up to 35% by weight, based in each case on the sum total of ethylene and interpolymerized ethylenically unsaturated carboxylic acid or acids. Examples of interpolymerizees include:

vinyl acetate,
one or more ethylenically unsaturated carboxylic esters, preferably of the formula III

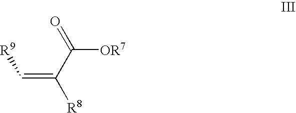

R⁷ is selected from C₁-C₁₀-alkyl, such as methyl, ethyl, n-propyl, iso-propyl, n-butyl, iso-butyl, sec-butyl, tert-butyl, n-pentyl, iso-pentyl, sec-pentyl, neo-pentyl, 1,2-dimethylpropyl, iso-amyl, n-hexyl, iso-hexyl, sec-hexyl, n-heptyl, n-octyl, n-nonyl, n-decyl; more preferably C₁-C₄-alkyl such as methyl, ethyl, n-propyl, iso-propyl, n-butyl, iso-butyl, sec-butyl and tert-butyl,
R⁸ is selected from hydrogen,
   C₁-C₁₀-alkyl, such as methyl, ethyl, n-propyl, iso-propyl, n-butyl, iso-butyl, sec-butyl, tert-butyl, n-pentyl, iso-pentyl, sec-pentyl, neo-pentyl, 1,2-dimethylpropyl, iso-amyl, n-hexyl, iso-hexyl, sec-hexyl, n-heptyl, n-octyl, n-nonyl, n-decyl; more preferably C₁-C₄-alkyl such as methyl, ethyl, n-propyl, iso-propyl, n-butyl, iso-butyl, sec-butyl and tert-butyl,
R⁹ is selected from hydrogen,
   C₁-C₁₀-alkyl, such as methyl, ethyl, n-propyl, iso-propyl, n-butyl, iso-butyl, sec-butyl, tert-butyl, n-pentyl, iso-pentyl, sec-pentyl, neo-pentyl, 1,2-dimethylpropyl, iso-amyl, n-hexyl, iso-hexyl, sec-hexyl, n-heptyl, n-octyl, n-nonyl, n-decyl; more preferably C₁-C₄-alkyl such as methyl, ethyl, n-propyl, iso-propyl, n-butyl, iso-butyl, sec-butyl and tert-butyl;
   COOH, COOCH₃, COOC₂H₅.

Most preferably, R⁸ is hydrogen or methyl and R⁹ hydrogen.

Most preferably, R⁸ is hydrogen or methyl and R⁹ hydrogen and R⁷ selected from methyl, ethyl, n-butyl and 2-ethylhexyl.

Ethylene copolymer (b) may advantageously be prepared by free-radically initiated copolymerization under high pressure conditions, for example in stirred high pressure autoclaves or in high pressure tubular reactors. Production in stirred high pressure autoclaves is preferred. Stirred high pressure autoclaves are known per se, a description is to be found in Ullmann's Encyclopedia of Industrial Chemistry, 5th edition, headwords: Waxes, volume A 28, pp. 146 ff., published by Chemie Weinheim, Basle, Cambridge, New York, Tokyo, 1996. Their length/diameter ratio ranges predominantly in intervals from 5:1 to 30:1 and preferably from 10:1 to 20:1. The similarly useful high pressure tubular reactors are likewise to be found in Ullmann's Encyclopedia of Industrial Chemistry, 5th edition, headwords: Waxes, volume A 28, pp. 146 ff., published by Chemie Weinheim, Basle, Cambridge, New York, Tokyo, 1996.

Suitable pressure conditions for the copolymerization are 500 to 4000 bar and preferably 1500 to 2500 bar. The reaction temperatures range from 170 to 300° C. and preferably from 200 to 280° C.

The copolymerization may be carried out in the presence of a regulator. Useful regulators include for example hydrogen or an aliphatic aldehyde or an aliphatic ketone of the general formula III

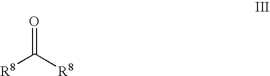

or mixtures thereof.

In formula III, the R⁸ radicals are the same or—particularly in the case of aldehydes—different and selected from
hydrogen;
C₁-C₆-alkyl such as methyl, ethyl, n-propyl, iso-propyl, n-butyl, iso-butyl, sec-butyl, tert-butyl, n-pentyl, iso-pentyl, sec-pentyl, neo-pentyl, 1,2-dimethylpropyl, iso-amyl, n-hexyl, iso-hexyl, sec-hexyl, more preferably C₁-C₄-alkyl such as methyl, ethyl, n-propyl, iso-propyl, n-butyl, iso-butyl, sec-butyl and tert-butyl;
C₃-C₁₂-cycloalkyl such as cyclopropyl, cyclobutyl, cyclopentyl, cyclohexyl, cycloheptyl, cyclooctyl, cyclononyl, cyclodecyl, cycloundecyl and cyclododecyl; preference is given to cyclopentyl, cyclohexyl and cycloheptyl.

In one particular embodiment, the R⁸ radicals are covalently bonded to each other to form a 4- to 13-membered ring. Thus, the two R⁸ radicals may together be for example: —(CH₂)₄—, —(CH₂)₅—, —(CH₂)₆, —(CH₂)₇—, —CH(CH₃)—CH₂—CH₂—CH(CH₃)— or —CH(CH₃)—CH₂—CH₂—CH₂—CH(CH₃)—.

Useful initiators for the free radical copolymerization include customary free radical initiators such as for example organic peroxides, oxygen or azo compounds. Mixtures of a plurality of free radical initiators are also suitable.

Suitable peroxides, selected from commercially available substances, are didecanoyl peroxide, 2,5-dimethyl-2,5-di(2-ethylhexanoylperoxy)hexane, tert-amyl peroxy-2-ethyl-hexanoate, dibenzoyl peroxide, tert-butyl peroxy-2-ethylhexanoate, tert-butyl peroxy-diethylacetate, tert-butyl peroxydiethylisobutyrate, 1,4-di(tert-butylperoxycarbonyl)-cyclohexane as isomeric mixture, tert-butyl perisononanoate 1,1-di(tert-butylperoxy)-3,3,5-trimethylcyclohexane, 1,1-di(tert-butylperoxy)cyclohexane, methyl isobutyl ketone peroxide, tert-butyl peroxyisopropyl carbonate, 2,2-di-tert-butylperox)butane or tert-butyl peroxyacetate;

tert-butyl peroxybenzoate, di-tert-amyl peroxide, dicumyl peroxide, the isomeric di(tert-butylperoxyisopropyl)benzenes, 2,5-dimethyl-2,5-di-tert-butylperoxyhexane, tert-butyl cumyl peroxide, 2,5-dimethyl-2,5-di(tert-butylperoxy)-hex-3-yne, di-tert-butyl peroxide, 1,3-diisopropylbenzene monohydroperoxide, cumene hydroperoxide or tert-butyl hydro-peroxide; or
dimeric or trimeric ketone peroxides, as known from EP-A 0 813 550.

Particularly suitable peroxides are di-tert-butyl peroxide, tert-butyl peroxypivalate, tert-butyl peroxyisononanoate or dibenzoyl peroxide or mixtures thereof. Azobisisobutyro-nitrile (“AIBN”) is an example of a useful azo compound. Free radical initiators are metered in amounts customary for polymerizations.

Numerous commercially available organic peroxides are admixed with so-called phlegmatizers before they are sold in order to make them better handleable. Examples of suitable phlegmatizers are white oil or hydrocarbons such as isododecane in particular.

In one embodiment of the present invention, ethylene copolymers (b) have a melt flow rate (MFR) in the range from 1 to 500 g/10 min, preferably in the range from 5 to 200 g/10 min and more preferably in the range from 7 to 50 g/10 min, measured at 160° C. and under a load of 325 g in accordance with German standard specification DIN 53735.

In one embodiment of the present invention, ethylene copolymers (b) have a kinematic melt viscosity ν in the range from 500 to 10 000 mm²/s and preferably in the range from 800 to 4000 mm²/s, measured in accordance with German standard specification DIN 51562.

In one embodiment of the present invention, the melting ranges of ethylene copolymers (b) are in the range from 60 to 115° C. and preferably in the range from 65 to 110° C., determined by DSC in accordance with German standard specification DIN 51007.

In one embodiment of the present invention, the density of ethylene copolymer (b) is in the range from 0.89 to 1.10 g/cm³ and preferably in the range from 0.92 to 0.94 g/cm³, determined in accordance with German standard specification DIN 53479.

Ethylene copolymer (b) may be an alternating copolymer or a block copolymer or preferably a random copolymer.

Ethylene copolymer (b) may with regard to interpolymerized ethylenically unsaturated carboxylic acid and/or ethylenically unsaturated C₄-C₁₀-dicarboxylic acid be present as a free acid or be partially or fully neutralized, for example with alkali metal such as for example sodium or potassium or with alkaline earth metal such as for example magnesium or calcium or with ammonia or organic amine, particularly C₁-C₄-mono-alkylamine, di-C₁-C₄-alkylamine, tri-C₁-C₄-alkylamine or tetra-C₁-C₄-alkylammonium. Similarly, any interpolymerized ethylenically unsaturated C₄-C₁₀-dicarboxylic anhydride may be present in hydrolyzed form.

In one embodiment of the present invention, up to 90 mol % and preferably up to 75 mol % of the carboxylic acid groups of ethylene copolymer (b) are neutralized with hydroxyalkylammonium particularly of the formula (C₁-C₄-Alkyl)ₓ(C₂-C₄-ω-hydroxy-alkyl)_{y}NH_{4-x-y}, where

x is an integer in the range from zero to three, preferably zero or one,
y is an integer in the range from one to four,
with the proviso that the sum of x and y does not exceed a value of four.

Preferred examples of C₁-C₄-ω-hydroxyalkyl are 3-hydroxypropyl, 4-hydroxybutyl and particularly 2-hydroxyethyl, hereinafter also referred to as hydroxyethyl.

Particularly preferred examples of hydroxyalkylammonium are N,N-dihydroxyethyl-ammonium, N-methyl-N-hydroxyethylammonium, N,N-dimethyl-N-hydroxyethyl-ammonium, N-methyl-N,N-dihydroxyethylammonium, N-n-butylhydroxyethyl-ammonium, N-n-butyl-N,N-dihydroxyethylammonium.

Further suitable organic amines for neutralizing are for example morpholine, imidazole, N4-amines, imidazoline, oxazolines, triazoles and fatty acid alkanolamines.

Suitable agents for neutralizing further include KOH, NaOH, Ca(OH)₂, NaHCO₃, Na₂CO₃, K₂CO₃ and KHCO₃.

Aqueous formulations used in the process of the present invention and comprising ethylene copolymer (b) preferably have an alkaline pH, for example a pH in the range from 7.5 to 14, preferably a pH of 8 or higher and more preferably a pH of 8.5 or higher.

In one embodiment of the present invention, polypropylene textile may be treated at a temperature in the range from 0° C. to 145° C. and preferably up to 130° C. To treat at temperatures in the range from 100 to 145° C., superatmospheric pressure has to be employed. To treat at temperatures in the range from 0 to 100° C., atmospheric pressure is suitable as well.

One embodiment of the present invention comprises treating polypropylene textile with an aqueous formulation that may also be referred to here as an aqueous liquor. To conduct the process of the present invention such that polypropylene textile to be treated is treated with an aqueous liquor, the wet pickup may be chosen such that a wet pickup in the range from 25% by weight to 95% by weight and preferably in the range from 60% to 90% by weight results through the process of the present invention.

One embodiment of the present invention comprises conducting the process of the present invention in common machines used for finishing textiles, examples being pad mangles. Preference is given to pad mangles having a perpendicular textile intake where the essential element is two squeeze rollers through which the polypropylene textile is led. Preferably aqueous formulation is introduced above the rollers and wets the polypropylene textile. The pressure causes the polypropylene textile to be squeezed off and ensures a constant add-on level. In other preferred pad mangles, polypropylene textile is initially led through a dip bath and subsequently upwardly through two squeeze rollers. In the latter case, the pad mangles are also referred to as those having a perpendicular textile intake from below. Pad mangles are described for example in Hans-Karl Rouette, “Handbuch der Textilveredlung”, Deutscher Fachverlag 2003, pages 618 to 620.

In one embodiment of the present invention, the process of the present invention may be carried out in the manner of an exhaust process, for example by spraying, nip-padding, kiss-roll or by printing out.

One embodiment of the present invention comprises conducting the process of the present invention in the manner of an exhaust process using a wet pickup in the range from 1 to 50% and preferably in the range from 20 to 40%.

In one embodiment of the present invention, the treatment of polypropylene textile may be followed by a thermal treatment, for example by drying at temperatures in the range from 30 to 100° C. or by thermal fixing at temperatures in the range of at least 100 and preferably at least 101° C. up to 150° C. and preferably up to 135° C.

In one embodiment of the present invention, thermal treatment may be carried out for a period in the range from 10 seconds to 30 minutes and preferably in the range from 30 seconds to 10 minutes.

One embodiment of the present invention comprises conducting two thermal treating steps at different temperatures, for example by drying in a first step at temperatures in the range from 30 to 100° C. for a period in the range from 10 seconds to 20 minutes and thereafter by fixing at temperatures in the range from 101 to 135° C. for a period in the range from 30 seconds up to 3 minutes.

In one preferred embodiment of the present invention, aqueous formulation used in the process of the present invention may comprise at least one silicone compound (a) and at least one ethylene copolymer (b).

In one embodiment of the present invention, aqueous formulation used in the process of the present invention may comprise one or more additives (d). Suitable additives (d) are for example organic solvents, organic solvents such as dimethyl sulfoxide (DMSO), N-methylpyrrolidone (NMP), N-ethylpyrrolidone (NEP), ethylene glycol, diethylene glycol, butyglycol, dibutylglycol and for example alkoxylated n-C₄-C₆-alkanol free of residual alcohol, preferably singly to 10-tuply, and more preferably 3- to 6-tuply ethoxylated n-C₄-C₆-alkanol free of residual alcohol. Residual alcohol here refers to the respectively nonalkoxylated n-C₄-C₆-alkanol.

The present invention further provides aqueous formulations comprising
(a) at least one silicone compound having at least one hydrophilic group or
(b) at least one ethylene copolymer obtainable by copolymerization of ethylene with at least one ethylenically unsaturated mono- or dicarboxylic acid or anhydride.

Silicone compound (a) and ethylene copolymer (b) are described above.

In one embodiment of the present invention, aqueous formulation in accordance with the present invention comprises:

0.1% to 20% by weight and preferably 0.5% to 10% by weight of silicone compound (a) or
1% to 25% by weight and preferably 2% to 25% by weight of ethylene copolymer (b).

In one embodiment of the present invention, aqueous formulation in accordance with the present invention comprises no colorants such as for example pigments, or dyes including disperse dyes.

Aqueous formulation of the present invention may comprise nought up to altogether 5% by weight of additives, preferably 0.5% to 3.5% by weight, based on entire aqueous formulation of the present invention.

The present invention further provides polypropylene textiles treated by the process of the present invention. Textiles of the present invention feature good and generally durable hydrophilicity, detectable for example by determining the sink depth of a drop of water. Furthermore, such polypropylene textiles of the present invention as are used as or for producing clothing are observed to give improved wear comfort.

In one embodiment of the present invention, polypropylene textiles of the present invention comprise 0.1% by weight to 5% by weight and preferably 0.5% by weight to 3% by weight of silicone compound (a) or 0.1% by weight to 5% by weight and preferably 0.5% by weight to 3% by weight of ethylene copolymer (b).

Polypropylene textiles of the present invention are very useful for example as hygiene nonwovens, as fire protection components for building materials such as for example mortar or concrete, as a constituent of sportswear, underwear, safety clothing or filters.

The present invention further provides building materials such as for example mortar or in particular concrete, comprising at least one polypropylene textile of the present invention, preferably in the form of microfibers or microfilaments. The distribution of textiles in accordance with the present invention in building materials in accordance with the present invention is excellent, and bonding between the two is good. In addition, building materials of the present invention, when installed in built structures such as buildings for example, are not as prone to spall at high temperatures. To produce building materials comprising at least one textile of the present invention, one possible procedure is for example for textile of the present invention to be introduced into conventional building material such as for example concrete or mortar and mixed in. To process, then, building material of the present invention such as for example concrete of the present invention can be cast by conventional methods, or building material of the present invention, in particular mortar of the present invention can be applied by conventional methods.

The present invention further provides hygiene nonwovens, clothing textiles such as sportswear, underwear or safety clothing and also geotextiles produced from or using textile of the present invention.

The present invention further provides hygiene nonwovens, for example for diapers or sanitary napkins, produced using textile of the present invention.

The invention is illustrated by examples.

### General Observations:

The following equipment was used in all cases:

Pad mangle: from Mathis, type No. HVF12085, contact pressure 1-3 bar. Contact pressure was in all cases adjusted such that the wet pickup (on weight of fabric) was 70 to 100%. The liquor was at room temperature, unless otherwise stated.
Dryer/fixer: continuous dryer from Mathis THN 12589
The sink time was determined using the TEGEWA drop test.

### I. Preparation of Silicone Compounds

### I.1 Preparation of Hydrophilic Silicone Compound (a.1)

480 g of an α,ω-dihydropolydimethylsiloxane with 0.055% by weight of Si-attached hydrogen and a water content of 50 weight ppm were mixed with 268 g of a polyether of the formula I.1

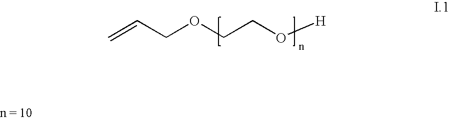

having a water content of 686 weight ppm and heated to 10° C. Thereafter, 0.14 g of a 2.7% by weight solution (based on elemental platinum) of a platinum-1,3-divinyl-1,1,3,3-tetramethyldisiloxane complex in α,ω-divinylpolydimethylsiloxane having a dynamic viscosity of 1000 mPa·s was added, determined at 25° C. The temperature rose by about 19° C., and a clear product formed. After stirring at 100 to 110° C. for one hour conversion of the Si-attached hydrogen was complete.

This was followed by the addition of 49.5 g of bis(dimethylaminopropyl)amine and 65 g of hexamethylenediisocyanate in succession and also of 50 mg of di-n-butyltin dilaurate. The mixture was stirred at 10° C. for 2 hours and cooled down to 70° C. 35 g of acetic acid and 225 g of diethylene mono-n-butyl ether were added to obtain (a.1) as a transparent brown oil having a kinematic viscosity of 4900 mm²/s, determined at 25° C., and an amine number of 0.47. The amine number is the number of ml of 1 N HCl needed to neutralize 1 g of (a.1).

Stirring 40 g of the solution described above into 60 g of water gave an emulsion having an amine number of 0.19.

### Preparation of a Silicone Compound (a.2)

245 g of an α,ω-dihydropolydimethylsiloxane with 0.055% by weight of Si-attached hydrogen and a water content of 50 weight ppm were mixed with 500 g of a polyether of the formula I.2

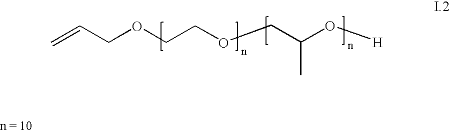

having an iodine number of 13.7 gl₂/100 gl.2 and a water content of 978 weight ppm and heated to 10° C. Thereafter, 0.14 g of a 2.7% by weight solution (based on elemental platinum) of a platinum-1,3-divinyl-1,1,3,3-tetramethyldisiloxane complex (as catalyst) in α,ω-divinylpolydimethylsiloxane having a dynamic viscosity of 1000 mPa·s was added, determined at 25° C. The temperature rose by about 6° C., whereupon the same amount of catalyst was added again, and a clear product formed. After stirring at 100 to 110° C. for one hour conversion of the Si-attached hydrogen was complete. An intermediate was obtained.

735 g of the intermediate described above were introduced as initial charge, followed by the addition of 25.5 g bis(dimethylaminopropyl)amine and 33.5 g of hexamethylene-diisocyanate in succession and also of 50 mg of di-n-butyltin dilaurate. The mixture was stirred at 10° C. for 2 hours and cooled down to 70° C. 17.5 g of acetic acid and 205 g of diethylene mono-n-butyl ether were added to obtain (a.2) as a transparent brown oil having a kinematic viscosity of 7800 mm²/s, determined at 25° C., and an amine number of 0.26.

Stirring 40 g of the solution described above into 60 g of water gave an aqueous emulsion of (a.2) having an amine number of 0.1.

### II. Production of Inventive Polypropylene Textiles

### II.1 Production of Inventive Textile T1

A woven fabric (100% of polypropylene, 150 g/m² areal weight) was pad-mangled with an aqueous formulation consisting of

30 g/l of aqueous emulsion of (a.1) of Example 1.1
1 g/l of 1-hexanol ethoxylate (5 mol of ethylene oxide/mol of n-hexanol), free of n-hexanol,
0.5 g/l of acetic acid,
the remainder being water.

This is followed by drying at 120° C. for 5 minutes to obtain inventive textile T1.

The sink time for a drop of distilled water was 8 seconds (untreated textile) or less than 0.5 seconds (inventive textile T1).

### II.1 Production of Inventive Textile T2

A polypropylene nonwoven (100% polypropylene, basis weight 90 g/m²) was pad-mangled with an aqueous formulation consisting of

150 g/l of ethylene copolymer (b.1) in aqueous dispersion, solids content 25%, neutralized with NH₃ (average particle diameter (number average) about 70 nm) having the following analytical data:

| | Methacrylic acid | Acid number | | |
|---|---|---|---|---|
| Ethylene content | content | [mg KOH/g | Tₘₑₗₜ | |
| [% by weight] | [% by weight] | (b.1)] | [° C.] | ρ [g/cm³] |
| 72.8 | 27.2 | 170 | 79.3 | 0.961 |

30 g/l of aqueous emulsion of (a.2) from 1.2
2 g/l of 1-hexanol ethoxylate (5 mol of ethylene oxide/mol of n-hexanol), free of n-hexanol,
0.5 g/l of acetic acid,
the remainder being water.

The wet pickup was about 70%. This was followed by drying at 100° C. for 5 minutes and subsequently by fixing at 110° C. for 1 minute to obtain inventive textile T2.

The sink time for a drop of water was >30 seconds (untreated textile) or respectively <0.5 seconds (inventive textile T2).

1-12. (canceled)

13. A process for treating a polypropylene textile, which comprises treating the polypropylene textile with an emulsifier-free aqueous formulation comprising
(a) at least one silicone compound obtainable by the reaction of a silicone compound having at least one NH group or at least one C—OH group with at least one aliphatic, cycloaliphatic or aromatic diisocyanate, or
(b) at least one ethylene copolymer obtainable by the copolymerization of ethylene with at least one ethylenically unsaturated mono- or dicarboxylic acid or anhydride.

14. The process according to claim 13 wherein the polypropylene textile comprises a thread-shaped or sheetlike structure composed of polypropylene.

15. The process according to claim 13 wherein said silicone compound (a) comprises a silicone compound obtainable by the reaction of a silicone compound having at least one NH group or at least one C OH per molecule with at least one aliphatic, cycloaliphatic or aromatic diisocyanate and one or more diols, triols, diamines, triamines or polyamines.

16. The process according to claim 13 wherein said ethylene copolymer (b) comprises a copolymer of ethylene with (meth)acrylic acid.

17. The process according to claim 13 wherein said formulation additionally comprises polyacrylate (c) which comprises a homopolymer of (meth)acrylic acid or a copolymer of acrylic acid with methacrylic acid, or a copolymer of (meth)acrylic acid with one or more C1C10-alkyl acrylates or with a polyethylene oxide ester of (meth)acrylic acid.

18. The process according to claim 13 wherein the emulsifier-free aqueous formulation comprises less than 0.1% by weight of cationic, anionic and nonionic emulsifier having a molecular weight of up to 400 g/mol, based on the entire aqueous formulation.

19. The process according to claim 13 wherein a thermal treatment is carried out following the treatment with said aqueous formulation.

20. A polypropylene textile treated by a process according to claim 13.

21. A hygiene nonwoven, an apparel textile or a geotextile comprising at least one textile according to claim 20.

22. A building material comprising at least one textile according to claim 20.

23. An aqueous formulation comprising
(a) at least one silicone compound obtainable by the reaction of a silicone compound having at least one NH group or at least one C—OH group with at least one aliphatic, cycloaliphatic or aromatic diisocyanate, or
(b) at least one ethylene copolymer having a melt flow rate (MFR) in the range from 1 to 500 g/10 minutes, measured at 160° C. and under a load of 325 g in accordance with German standard specification DIN 53735, said at least one ethylene copolymer being obtainable by the copolymerization of ethylene with at least one ethylenically unsaturated mono- or dicarboxylic acid or anhydride.
